(19) United States
(12) Patent Application Publication
(10) Pub. No.: US 2005/0214803 A1
(43) Pub. Date: Sep. 29, 2005

(54) High-density amine-functionalized surface

(75) Inventor: Guo Bin Wang, Malden, MA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: SRU BIOSYSTEMS, LLC, Woburn, MA (US)

(21) Appl. No.: 10/983,511

(22) Filed: Nov. 8, 2004

Related U.S. Application Data
(60) Provisional application No. 60/517,847, filed on Nov. 6, 2003.

(51) Int. Cl. C12Q 1/68; G01N 33/53; C12M 1/34; B05D 3/00; G01N 33/543

(52) U.S. Cl. 435/6; 435/287.1; 435/7.5; 427/2.11

(57) ABSTRACT

A method for providing amine functional groups on a surface for binding proteins, peptides, DNAs, cells, small molecules, and other chemical or biological molecules that are of interests in the areas of proteomic, genomic, pharmaceutical, drug discovery, and diagnostic studies.

This application claims priority to U.S. Provisional Application No. 60/517,847, filed Nov. 6, 2003, which is hereby incorporated by reference herein in its entirety, including the drawings.

### BACKGROUND OF THE INVENTION

1. Field of the Invention

This invention relates to a surface having amine functional groups useful for attaching chemical or biological molecules. The invention also relates to methods of generating high performance surface chemistry using grafting of functional polymers, for example, to immobilize covalently attached biomolecules for subsequent biomolecule interaction detection.

2. Background of the Invention

With the completion of the sequencing of the human genome, one of the next grand challenges of molecular biology will be to understand how the many protein targets encoded by DNA interact with other proteins, small molecule pharmaceutical candidates, and a large host of enzymes and inhibitors. See e.g., Pandey & Mann, “Proteomics to study genes and genomes,” Nature, 405, p. 837-846, 2000; Leigh Anderson et al., “Proteomics: applications in basic and applied biology,” Current Opinion in Biotechnology, 11, p. 408-412, 2000; Patterson, “Proteomics: the industrialization of protein chemistry,” Current Opinion in Biotechnology, 11, p. 413-418, 2000; MacBeath & Schreiber, “Printing Proteins as Microarrays for High-Throughput Function Determination,” Science, 289, p. 1760-1763, 2000; De Wildt et al., “Antibody arrays for high-throughput screening of antibody-antigen interactions,” Nature Biotechnology, 18, p. 989-994, 2000. To this end, tools that have the ability to simultaneously identify and/or quantify many different biomolecular interactions with high sensitivity will find application in pharmaceutical discovery, proteomics, and diagnostics. Further, for these tools to find widespread use, they must be simple to use, inexpensive to own and operate, and applicable to a wide range of analytes that can include, for example, polynucleotides, peptides, small proteins, antibodies, and even entire cells.

The immobilization of target molecules onto support surfaces has become an important aspect in the development of biological assays. Generally, biological assays are carried out on the surfaces of microwell plates, microscope slides, tubes, silicone wafers or membranes. The target molecules are covalently immobilized on the surface using coupling reactions between the functional groups on the surface and the functional groups of the molecules. One of popular surface functionalization techniques on glass surface is silanization using functional silanes. Silane, Silicones, and Metal-Organics, p. 88, published by Gelest Inc., Tullytown, Pa. (2000). GAPS II coated slides manufactured by Corning Inc. (Corning, N.Y.), Arryit™ SuperAmine slides supplied by TeleChem International, Inc (Sunnyvale, Calif.), SILANE-PREP™ amine-functionalized slides provided by Sigma Diagnostics (St Louis, Mo.) and others are examples of available biological assay surfaces in microscope slide format. The SuperAmine slide is claimed to provide 5×10¹² amine groups per mm². As another example, amide groups that have been derivatized amidine on a Nylon support are used to immobilize DNA and RNA probes in hybridization assays to detect specific polynucleotide sequences. See U.S. Pat. No. 4,806,546. Products in formats of microwell plates and tubes, including NucleoLink™ and CovaLink™ provided by Nalge Nunc International (Rochester, N.Y.), are available only on polymeric support surfaces. The CovaLink™ products provide a secondary amine surface at approximately 10¹² groups per mm² of surface area. Secondary amines show a lower reactivity than primary amines in many conjugation reactions. See, Loudon, G. Marc, Organic Chemistry, 3d ed., The Benjamin/Cummings Publishing, Redwood City, Calif. (1995).

There are numerous known methods for chemically functionalizing the surfaces of materials, such as silicon, glass or gold for example. Surface functionalization is of great interest, as it often leads to expanded applications for the surface, whereby enhanced binding and analysis of various molecules to the surface becomes possible, relative to a surface with a non-chemically functionalized surface. The type, quantity, and quality of a chemical functionalization coating on a surface determine the covalent strength and capacity of the surface to bind a particular analyte. It is highly desirable that the coating itself not be easily washed away or degraded after multiple uses. Amine functional groups coated on a surface have been shown to provide a versatile platform for detecting biomolecules. These groups can capture biomolecules through physical attraction, such as electrostatic interaction, for example, or chemical binding. Such chemical binding can be achieved directly or indirectly (i.e. through a chemical linker). Many homobifunctional or heterobifunctional linkers are known in the field. A simple method for coating a surface with amine is to directly expose the cleaned surface to polylysine. An example is a glass slide surface used for microarray printing. This type of surface, however, has been shown to be unstable after multiple uses. An alternative to coating a surface with amines is to covalently attach amine-coating molecules to the surface, such as attaching silanes on glass or thiols on gold, both of which are well known.

Various aminoalkylsilane reagents have been used to coat silicon- or glass-based surfaces with amine groups. Processes used in coating such surfaces include the use of a variety of silane reagents, solvents, and different physical treatment procedures. Further, to test the presence of a chemical group on a surface, many methods including radioactive, colorimetric, fluorescence, XPS, FTIR, AFM and others have been used. Sensitivity is an important tissue when selecting the appropriate method for surface testing. Generally speaking, there is neither a standard industry procedure to chemically coat a biosensor sensor surface, nor a standardized testing method for detecting the presence or quantity of a particular chemical moiety on such a biosensor.

In the past, significant difficulty has been encountered in preparing chemically coated inert plastic-based surfaces. Attempting to chemically coat some plastic surfaces often leads to undesirable degradation, i.e., the plastic dissolves, is etched or is structurally corrupted. Further, in many cases, coating an inert plastic-based surface has not been practical, as the chemical coating layer does not adequately adhere to the surface and is easily washed away after multiple uses, particularly for hydrophilic polymer coating. With respect to amine-functionalized surfaces, the processes for preparing the surfaces have various undesirable compositional and/or processing limitations such as incompatible reagents, undesirably long reaction times, Or necessarily elevated curing temperatures that would alter or degrade the plastic-based surface.

Further, it has been difficult to characterize the functional groups on the surface, qualitatively and/or quantitatively. To verify the presence of amine groups, the current colorimetric and fluorescence methods usually deal with samples that are in solution where detection is much more sensitive than samples on a dry surface. To perform surface characterization of chemical groups that are less than 10 angstrom thick on a dry and not-totally-flat surface has also been proven a difficult task.

For example, biosensors have been developed to detect a variety of biomolecular complexes including oligonucleotides, antibody-antigen interactions, hormone-receptor interactions, and enzyme-substrate interactions. In general, biosensors consist of two components: a highly specific recognition element and a transducer that converts the molecular recognition event into a quantifiable signal. Signal transduction has been accomplished by many methods, including fluorescence, interferometry (Jenison et al., “Interference-based detection of nucleic acid targets on optically coated silicon,” Nature Biotechnology, 19, p. 62-65; Lin et al, “A porous silicon-based optical interferometric biosensor,” Science, 278, p. 840-843, (1997)), and gravimetry (A. Cunningham, Bioanalytical Sensors, John Wiley & Sons (1998)).

Of the optically-based transduction methods, direct methods that do not require labeling of analytes with fluorescent compounds are of interest due to the relative assay simplicity and ability to study the interaction of small molecules and proteins that are not readily labeled. Direct optical methods include surface plasmon resonance (SPR) (Jordan & Corn, “Surface Plasmon Resonance Imaging Measurements of Electrostatic Biopolymer Adsorption onto Chemically Modified Gold Surfaces,” Anal. Chem., 69:1449-1456 (1997)), grating couplers (Morhard et al., “Immobilization of antibodies in micropatterns for cell detection by optical diffraction,” Sensors and Actuators B, 70, p. 232-242, (2000)), ellipsometry (Jin et al., “A biosensor concept based on imaging ellipsometry for visualization of biomolecular interactions,” Analytical Biochemistry, 232, p. 69-72, (1995)), evanescent wave devices (Huber et al., “Direct optical immunosensing (sensitivity and selectivity),” Sensors and Actuators B, 6, p. 122-126, (1992)), and reflectometry (Brecht & Gauglitz, “Optical probes and transducers,” Biosensors and Bioelectronics, 10, p. 923-936, (1995)). Theoretically predicted detection limits of these detection methods have been determined and experimentally confirmed to be feasible down to diagnostically relevant concentration ranges. However, to date, these methods have yet to yield commercially available high-throughput instruments that can perform high sensitivity assays without any type of label in a format that is readily compatible with the microtiter plate-based or microarray-based infrastructure that is most often used for high-throughput biomolecular interaction analysis.

Chemical and biological molecules, such as those participating in biological assays, have steric structure in assay mediums. When immobilized on a solid surface, the molecules conformation may be obstructed. When a high density of the chemical or biological molecules is immobilized on a two-dimensional-support surface, steric crowding occurs. Southern, E. et al., Nature Genetics Supp. 21:5 (1999). The issue of steric crowding or accessibility largely influences the interaction of the chemical or biological molecule. This is particularly true for many large-size molecules. For example, Gray and coworkers have reported that oligonucleotide bases appear to dissolve enough from support surfaces to eliminate steric hindrance when ammonia is used to deprotect the oligonucleotide, resulting in an improved hybridization signal being observed. Gray, D E, et al., Langmuir, 13:2833 (1997); Matson, R S, Anal. Biochem. 223(1):110 (1995). Shchepinov et al. have demonstrated that adding spacers between immobilized oligonucleotides and a solid support surface significantly improved hybridization signals. Shchepinov, M. S., et al., Nucleic Acids Res., 25(6):1155-61 (1997).

In order to increase the density of functional groups on a support surface, and to reduce steric hindrance, chemically functional polymers have been used to provide three-dimensional matrixes at the top of the support surface. For example, the three-dimensional protein microarray substrate HydroGel™ coated slides, provided by Perkin Elmer Life Science (Boston, Mass.), provides a highly swellable polymer matrix for protein interaction. This polymeric matrix has a 2 μm thickness when dry and up to 90 μm thickness when fully hydrated. Wang, G. B., et al., Nucleic Acids Res. (in preparation). 3D-Link™ supplied by Arnersham Biosciences (Piscataway, N.J.) is also an attempt to provide a three-dimensional polymer microarray substrate. However, the network structure of the crosslinked polymer matrix limits the accessibility of the large-size biomolecules. U.S. Pat. No. 6,413,722, incorporated herein by reference. Reversed-phase surface polymerization can be used to grow non-crosslinked “brush” polymer structure even on most inert polymeric surfaces in aqueous solution through free radical transferring. Wang, G. B., et al., 6^{th} World Biomaterials Congress, Hawaii (2000); U.S. Pat. No. 6,358,557, incorporated herein by reference. The various functionalities and chemical functional group density can be readily obtained by adding functional free radical-polymerizable monomers or mixtures. However, it requires an organic polymer surface or polymeric primer on an inorganic surface. “Brush” polymeric surfaces are also built using free radical polymerization initiated by radical-generating surface on glass silanized with initiator-containing silane. E.P.O. Patent 1,176,423. The synthesis of the silanes is critical for this process. Amine-containing polymers have been covalently attached on amino-silanized glass surface using a coupling agent cyanuric chloride through multi-step reaction. However, cyanuric chloride activation has to be carried out in anhydrous solution, U.S. Pat. No. 6,413,722, and it is limited in process. Therefore, there remains a need in the art to address this issue.

### SUMMARY OF THE INVENTION

In one embodiment, the invention provides for a method for preparing a high-density amine-functionalized surface. The method includes:

(a) treating a surface with epoxy silane to form an epoxy-functional surface; and

(b) attaching one or more amine-containing polymers to the epoxy-functional surface by adding a solution comprising one or more amine-containing polymers to the epoxy-functional surface under conditions where one or more amine-containing polymers react with the epoxy-functional surface; whereby a high-density amine-functionalized surface is formed. Further, the surface can be plastic.

In another embodiment of the invention, the method comprises the step of covalently attaching one or more chemical or biological molecules to the one or more amine-containing polymers attached to the surface. The chemical or biological molecules can include proteins, peptides, polypeptides, nucleotides, polynucleotides, small molecules, biotin, cells, fractionated cells, cell extracts, cell fractions, and parts of cells. Further, the protein can be an enzyme, an antibody, avidin, streptavidin, or a peptide. Further, the chemical or biological molecule can be a small molecule. The small molecule can be biotin.

A further embodiment of the invention includes a biosensor comprising a high-density amine-functionalized surface. The biosensor can be an optical sensor, such as a colorimetric resonant biosensor. Alternatively, the biosensor can be an acoustic biosensor or an electric biosensor. Further, the surface can be plastic.

The high-density amine-functionalized surface can include one or more amine-containing polymers that are the same or that are different. The one or more amine-containing polymers may contain primary amines, secondary amines, or both. The amine-containing polymers may be polyethylenimine or polyvinylamine.

A further embodiment of the invention includes a high-density amine-functionalized polymeric matrix, comprising one or more amine-containing polymers covalently attached to a surface through a functional epoxy, wherein the amine-containing polymers are the same or different, and wherein the amine-containing polymers comprise two or more amine groups. Alternatively, the amine-containing polymers comprise three or more amine groups.

A further embodiment of the invention includes method of immobilizing biomolecules on a surface, comprising contacting biomolecules with a high-density amine-functionalized surface created by: (a) treating a surface with epoxy silane to form an epoxy-functional surface; and (b) attaching one or more amine-containing polymers to the epoxy-functional surface by adding a solution comprising one or more amine-containing polymers to the epoxy-functional surface under conditions where one or more amine-containing polymers react with the epoxy-functional surface; whereby the biomolecules are immobilized.

Another embodiment of the invention includes a biosensor comprising a high-density amine-functionalized surface, wherein the high-density amine-functionalized surface is prepared by the method comprising:

(a) treating a surface with epoxy silane to form an epoxy-functional surface; and

(b) attaching one or more amine-containing polymers to the epoxy-functional surface by adding a solution comprising one or more amine-containing polymers to the epoxy-functional surface under conditions where one or more amine-containing polymers react with the epoxy-functional surface; whereby a high-density amine-functionalized surface is formed. Further, the biosensor can be an optical sensor, a colorimetric resonant biosensor, and acoustic biosensor, or an electric biosensor. Further, the surface can be plastic.

### BRIEF DESCRIPTION OF THE FIGURES

FIG. 1 is a schematic diagram of various embodiments of an optical grating structure used for a colorimetric resonant reflectance biosensor. n_{substrate} represents substrate material. n₁ represents the refractive index of a cover layer. n₂ represents the refractive index of a one- or two-dimensional grating. n_{bio} represents the refractive index of one or more specific binding substances. t₁ represents the thickness of the cover layer. t₂ represents the thickness of the grating. t_{bio} represents the thickness of the layer of one or more specific binding substances.

FIG. 2 shows a grafting reaction by which an amine containing polymer is attached to an epoxy surface.

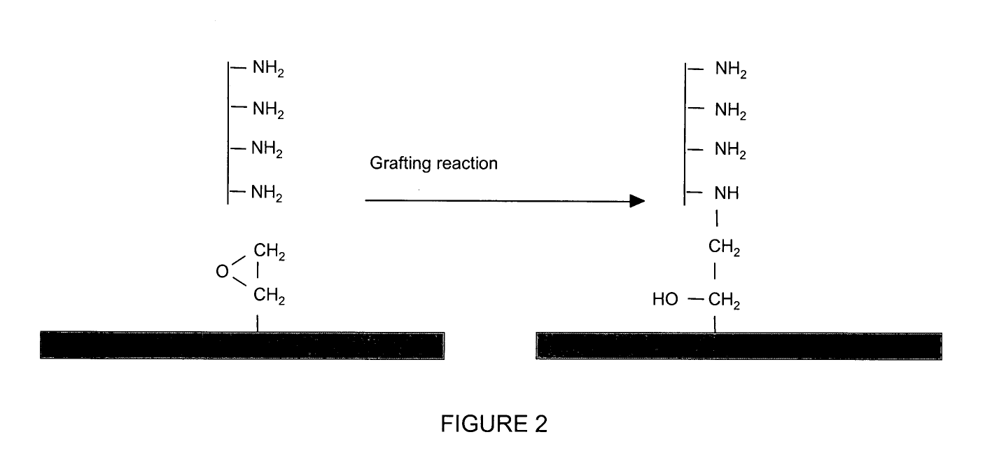

FIG. 3 shows the amine densities, represented as pmol/mm², of amine groups on different surfaces.

FIG. 4 shows the polyethylenimine (“PEI”) thickness, represented in angstroms, of samples that were either centrifuged dried or lyophilized, after the grafting reaction of PEI of five concentrations with epoxy surface in aqueous mediums.

FIG. 5 shows the detection response of the amount of molecule attached to the surface, the PWV shift, for the three identified groups.

FIG. 6 shows the response of streptavidin binding.

FIG. 7 shows the kinetic curves and the endpoints for SA immobilization on the treated surfaces.

FIG. 8 shows the biotin response to immobilized SA on the surface.

### DETAILED DESCRIPTION OF THE INVENTION

Amine coated surfaces are useful for binding chemical or biological molecules such as proteins, peptides, polypeptides, nucleotides, polynucleotides, small molecules, biotin, cells, fractionated cells, cells extracts, cell fractions, parts of cells and other chemical or biological molecules that are of interest in the areas of, for example, proteomics, genomics, pharmaceuticals, drug discovery, and diagnostic studies. For example, biosensors can be amine-coated to bind chemical or biological molecules that are of interest. The invention is directed to a high-density amine functionalized surface and a process for providing the high density of amine functional groups on the surface. The invention can provide a high density of functional amine binding sites using chemical reagents that do not alter or degrade plastic surfaces, such as those used with a plastic biosensor structure.

The methods of this invention provide, inter alia, methods of tethering covalently an amine-containing polymer onto an epoxy surface using a graft reaction between an amine group and epoxy group. The polymers of this invention contain more than one amine group. The polymers can contain primary amines, secondary amines, or both primary and secondary amines.

As used herein, amine refers to both primary amines having the formula —NH₂ that may be attached directly or through a linking molecule to the surface, as well as secondary amines. An amine-coated surface or an amine-functionalized surface refer to a surface which provides amine groups available for chemical modification, such as the attachment of chemical or biological molecules, either directly or indirectly. Indirect attachment refers to the attachment of chemical or biological molecules through a chemical linker as is well known in the art.

Plastic-based biosensors, or plastic biosensors, refer to those biosensors that contain a plastic grating or sensor surface, a plastic support for the grating, also referred to as a substrate, and/or other plastic components. Such biosensors are susceptible to degradation as the result of reaction conditions used to functionalize the surfaces of the biosensors. Plastics having optical qualities are preferred. The plastic can be clear and transparent without any particulate and can be capable of providing a smooth, flat finish. As an example, a biosensor can include a polyester substrate that supports an acrylic polymer grating layer. Other non-limiting examples of plastics include polyesters and polyurethanes. However, any plastic that provides optical qualities for use in a biosensor may be used. In another example, the grating surface is plastic, such that the plastic serves as both the substrate and the grating.

An amine-functionalized surface refers to a surface having a coating through which chemical and biological molecules may be attached. For example, an amine-functionalized surface can refer to, but is not limited to, a sensor surface of a plastic-based biosensor having a coating of a high refractive index material. Such high refractive index materials include, for example, silicon nitride, zinc sulfide, titanium dioxide or tantalum oxide. Optionally, a silicon oxide layer can be coated on the high refractive index material prior to surface functionalization. Either the high refractive index material or the silicon oxide can be functionalized with amine functional groups for attachment of chemical and biological molecules. The reagents used to amine functionalize the grating surface coated with the high refractive index material must be compatible with the grating material and the substrate material, whether they are acrylic polymers or other plastic. While the grating is coated with the high refractive index material, which provides some protection of the grating material from the reagents used to amine functionalize the surface, the opposite side of the grating may still be exposed during the functionalization process. Likewise, when the grating is bound to a substrate, the opposite side of the substrate may be exposed to the activation reagents. Also, imperfections in the coating of the high refractive index material on the grating surface may result in areas of the upper side of the grating surface exposed. Thus, the materials of the various layers and the adhesion between layers should remain intact during functionalization and any subsequent assay procedures.

An amine-functionalized surface of a biosensor refers to plastic-based biosensors, as well as biosensors that are not plastic based. For example, a biosensor includes a titanium oxide-coated sensor, or additional sensors with high refractive index, low index of absorption coating or covering for the top layer and for the base material construction. In addition, silicon dioxide, in all of its various physical forms, or other material with low index of absorption and low refractive index, are contemplated. These biosensors are meant to be exemplary, and are not limiting of biosensors that have an amine-functionalized surface.

Subwavelength Structured Surface (SWS) Biosensor

In one embodiment of the invention, a subwavelength structured surface (SWS) is used to create a sharp optical resonant reflection at a particular wavelength that can be used to track with high sensitivity the interaction of chemical or biological materials, such as specific binding substances or binding partners or both. A colorimetric resonant diffractive grating surface acts as a surface-binding platform for specific binding substances. Like ellipsometry, SPR, and reflectance spectrometry, this method utilizes a change in the refractive index upon a surface to determine when a chemically bound material is present within a specific location.

Subwavelength structured surfaces are an unconventional type of diffractive optic that can mimic the effect of thin-film coatings. (Peng & Morris, “Resonant scattering from two-dimensional gratings,” J. Opt. Soc. Am. A, Vol. 13, No. 5, p. 993, May; Magnusson, & Wang, “New principle for optical filters,” Appl. Phys. Lett., 61, No. 9, p. 1022, August, 1992; Peng & Morris, “Experimental demonstration of resonant anomalies in diffraction from two-dimensional gratings,” Optics Letters, Vol. 21, No. 8, p. 549, April, 1996). A SWS structure contains a surface-relief, one-dimensional or two-dimensional grating in which the grating period is small compared to the wavelength of incident light so that no diffractive orders other than the reflected and transmitted zeroth orders are allowed to propagate. See U.S. patent application Ser. Nos. 10/059,060 and 10/058,626, incorporated by reference in their entirety. A SWS surface narrowband filter can comprise a one-dimensional or two-dimensional grating sandwiched between a substrate layer and a cover layer that fills the grating grooves. Optionally, a cover layer is not used. When the effective index of refraction of the grating region is greater than the substrate or the cover layer, a guided mode resonant effect occurs. When a filter is designed properly, the one-dimensional or two-dimensional grating structure selectively couples light at a narrow band of wavelengths. The light undergoes scattering, and couples with the forward- and backward-propagating zeroth-order light. The guided mode resonant effect occurs over a highly localized region of approximately 3 microns from the point that any photon enters the structure. Because propagation of guided modes in the lateral direction are not supported, a waveguide is not created.

The reflected or transmitted color of this structure can be modulated by the addition of molecules such as specific binding substances or binding partners or both to the upper surface of the cover layer or the one-dimensional or two-dimensional grating surface. The added molecules increase the optical path length of incident radiation through the structure, and thus modify the wavelength at which maximum reflectance or transmittance will occur.

In one embodiment, a biosensor, when illuminated with white light, is designed to reflect only a single wavelength. When specific binding substances, such as chemical and biological molecules, are attached to the surface of the biosensor, the reflected wavelength (color) is shifted due to the change of the optical path of light that is coupled into the grating. By linking specific binding substances to a biosensor surface, complementary binding partner molecules can be detected without the use of any kind of fluorescent probe or particle label. The detection technique is capable of resolving changes of, for example, ˜0.1 nm thickness of protein binding, and can be performed with the biosensor surface either immersed in fluid or dried.

A detection system consists of, for example, a light source that illuminates a small spot of a biosensor at normal incidence through, for example, a fiber optic probe, and a spectrometer that collects the reflected light through, for example, a second fiber optic probe also at normal incidence. Because no physical contact occurs between the excitation/detection system and the biosensor surface, no special coupling prisms are required and the biosensor can be easily adapted to any commonly used assay platform including, for example, microtiter plates and microarray slides. A single spectrometer reading can be performed in several milliseconds, thus it is possible to quickly measure a large number of molecular interactions taking place in parallel upon a biosensor surface, and to monitor reaction kinetics in real time.

This technology is useful in applications where large numbers of biomolecular interactions are measured in parallel, particularly when molecular labels would alter or inhibit the functionality of the molecules under study. High-throughput screening of pharmaceutical compound libraries with protein targets, and microarray screening of protein-protein interactions for proteomics are examples of applications that require the sensitivity and throughput afforded by the compositions and methods of the invention.

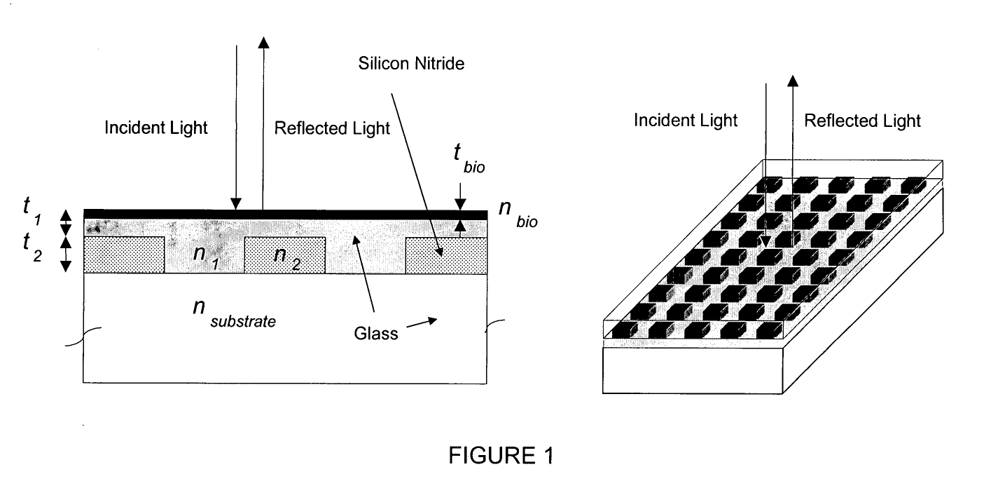

A schematic diagram of an example of a SWS structure is shown in FIG. 1. In FIG. 1, n_{substrate} represents a substrate material. n₁ represents the refractive index of an optional cover layer. n₂ represents the refractive index of a two-dimensional grating. N_{bio} represents the refractive index of one or more specific binding substances. t₁ represents the thickness of the cover layer above the two-dimensional grating structure. t₂ represents the thickness of the grating. t_{bio} represents the thickness of the layer of one or more specific binding substances. In one embodiment, n₂>n₁. (see FIG. 1). Layer thicknesses (i.e. cover layer, one or more specific binding substances, or a grating) are selected to achieve resonant wavelength sensitivity to additional molecules on the top surface. The grating period is selected to achieve resonance at a desired wavelength. The structures can be fabricated from glass and silicon nitride dielectric materials. Alternatively, structures may be formed from embossed plastic with an appropriate dielectric cover layer.

One embodiment of the invention provides a SWS biosensor. A SWS biosensor comprises a one-dimensional or two-dimensional grating, a substrate layer that supports the grating, and one or more specific binding substances immobilized on the surface of the grating opposite-of the substrate layer.

A one-dimensional or two-dimensional grating can be comprised of a material, including, for example, zinc sulfide, titanium dioxide, tantalum oxide, and silicon nitride. A cross-sectional profile of the grating can comprise any periodically repeating function, for example, a “square-wave.” A grating can be comprised of a repeating pattern of shapes selected from the group consisting of continuous parallel lines squares, circles, ellipses, triangles, trapezoids, sinusoidal waves, ovals, rectangles, and hexagons. A sinusoidal cross-sectional profile is preferable for manufacturing applications that require embossing of a grating shape into a soft material such as plastic, or replicating a grating surface into a material such as epoxy. In one embodiment of the invention, the depth of the grating is about 0.01 micron to about 1 micron and the period of the grating is about 0.01 micron to about 1 micron.

A SWS biosensor can also comprise a one-dimensional linear grating surface structure, i.e., a series of parallel lines or grooves. A one-dimensional linear grating is sufficient for producing the guided mode resonant filter effect. While a two-dimensional grating has features in two lateral directions across the plane of the sensor surface that are both subwavelength, the cross-section of a one-dimensional grating is only subwavelength in one lateral direction, while the long dimension can be greater than wavelength of the resonant grating effect. A one-dimensional grating biosensor can comprise a high refractive index material that is coated as a thin film over a layer of lower refractive index material with the surface structure of a one-dimensional grating. Alternatively, a one dimensional grating biosensor can comprise a low refractive index material substrate, upon which a high refractive index thin film material has been patterned into the surface structure of a one-dimensional grating. The low refractive index material can be glass, plastic, polymer, or cured epoxy. The high refractive index material must have a refractive index that is greater than the low refractive index material. The high refractive index material can be zinc sulfide silicon nitride, tantalum oxide, titanium dioxide, or indium tin oxide, for example.

In one embodiment, a SWS structure is used as a microarray platform by, for example, building a grating surface that is the same size as a standard microscope slide and placing microdroplets of high affinity chemical receptor reagents onto an x-y grid of locations on the grating surface. Alternatively, the SWS structure is built to be the same size as a standard microtiter plate, and incorporated into the bottom surface of the entire plate. When the chemically functionalized surface, for example the microarray/microtiter plate, is exposed to molecules, such as an analytes, the molecules will be preferentially attracted to locations that have high affinity. As a result, some surface locations gather additional material, and other surface locations do not. The surface locations that attract additional material can be determined by measuring the shift in resonant wavelength within each individual surface location, such as each individual microarry/microtiter surface location. Thus, for example, the amount of bound molecules, such as analytes, in the sample and the chemical affinity between receptor reagents and the molecules can be determined by measuring the extent of the shift of the resonant wavelength.

In one embodiment of the invention, an interaction of a first molecule with a second test molecule can be detected. A SWS biosensor as described above is used; however, there are no specific binding substances immobilized on its surface. Therefore, the biosensor comprises a one- or two-dimensional grating, a substrate layer that supports the one- or two-dimensional grating, and optionally, a cover layer. As described above, when the biosensor is illuminated a resonant grating effect is produced on the reflected radiation spectrum, and the depth and period of the grating are less than the wavelength of the resonant grating effect.

To detect an interaction of a first molecule with a second test molecule, a mixture of the first and second molecules is applied to a distinct location on a biosensor. A distinct location can be one spot or well on a biosensor or can be a large area on a biosensor. A mixture of the first molecule with a third control molecule is also applied to a distinct location on a biosensor. The biosensor can be the same biosensor as described above, or can be a second biosensor. If the biosensor is the same biosensor, a second distinct location can be used for the mixture of the first molecule and the third control molecule. Alternatively, the same distinct biosensor location can be used after the first and second molecules are washed from the biosensor. The third control molecule does not interact with the first molecule and is about the same size as the first molecule. A shift in the reflected wavelength of light from the distinct locations of the biosensor or biosensors is measured. If the shift in the reflected wavelength of light from the distinct location having the first molecule and the second test molecule is greater than the shift in the reflected wavelength from the distinct location having the first molecule and the third control molecule, then the first molecule and the second test molecule interact. Interaction can be, for example, hybridization of nucleic acid molecules, specific binding of an antibody or antibody fragment to an antigen, and binding of polypeptides. A first molecule, second test molecule, or third control molecule can be, for example, a nucleic acid, polypeptide, antigen, polyclonal antibody, monoclonal antibody, single chain antibody (scFv), F(ab) fragment, F(ab′)₂ fragment, Fv fragment, small organic molecules cell, virus, and bacteria.

Amine-Functionalized Biosensors

After a layer of high refractive index material, such as silicon nitride, is coated on the structure, such as a plastic structure, the device is prepared for use as a sensor by the attachment of amine-functional groups on the surface of the high refractive index material. Plastic-based biosensors can be degraded (i.e. structure or composition change on the sensor) during the chemical modification that provides amine functional groups on its surface. To avoid such degradation, the present invention provides for a process for amine surface functionalization of a biosensor using reagents that are compatible with the plastic of the biosensor. After a high refractive index material has been deposited on the grating surface of the plastic biosensor, the sensor may be stored or may be used directly for functionalization. The sensor may be subjected to a cleaning step using wet (e.g. cleaning using a liquid, such as solvent) or dry (e.g,. UV ozone or plasma) methods prior to the amine functionalization procedure. In one embodiment, the amine functionalization procedure includes (a) exposing a plastic colorimetric resonant biosensor to an alcoholic silane solution, and then (b) rinsing the exposed plastic colorimetric resonant biosensor with an alcohol. When the biosensor is dried, the grating surface contains amine functional groups, i.e., —NH₂ groups.

In one aspect of the invention, the silane solution includes a 3-aminopropyltriethoxysilane and an alcohol, such as ethanol or other suitable low molecular weight alcohol. Likewise any suitable low molecular weight alcohol may be used to rinse the biosensor. An example of coating the plastic biosensor with amine is first exposing the sensor to a solution containing 3-aminopropyltriethoxysilane and ethanol, then briefly rinsing the sensor in ethanol, and finally drying the sensor. The concentration of the 3-aminopropylsilane in ethanol may be adjusted such that the concentration of the 3-aminopropylsilane is from about 1% to about 15% in ethanol. In addition, the ethanol may be about 90%-100% (volume/volume, adjusted with water). The drying step may be done in an oven at about, 70° C. for 10 min for example. The drying may be performed at higher temperatures, provided the temperature is selected such that biosensor degradation does not occur.

In accordance with the invention, numerous suitable solvents, concentrations, reaction times, and curing/incubation times may be utilized. Contemplated variations of the invention includes the type of surface, the silane reagent (other silane such as 3-aminopropyltrimethoxysilane, etc.), the silane concentration, the coating solvent or a combination of solvents (e.g. ethanol and water), the coating reaction time, the rinse solvent or a combination of solvents (e.g. ethanol and water), the curing time, and the curing temperature.

Surface Treatment

In one embodiment of the invention, the biosensor surface can be modified by chemical treatment. For example, the surface can be treated with a solution by immersing the surface in the solution. Alternatively, gas-phase treatment, including chemical vapor or atomization deposition can also be used for a coating of the surface. Gas-phase treatment can be used to ensure a conformal coating of the geometrically non-flat surface. Such a coating can be used in a step of silanizing a surface, or for the addition of other organic materials to a surface. Other methods by which a surface can be treated will be recognized by those skilled in the art.

Treatment by plasma can be commonly used prior to the gas-phase coating processes. The plasma treatment can remove most contamination on the surface and activate some of the surfaces to improve the adhesion of the subsequent gas-phase coating process.

The gas-phase coating process can be used to add chemical functionality and minimize adsorbed moisture, organic contaminants, and low molecular weight material, on the surface of polymer films. The gas-phase coating has advantages including, but not limited to, the uniform treatment of surfaces, no backside treatment when polymer films are treated, no pin-holes when treating porous materials. Such coating services useful in this invention include but are not limited services provided by Sigma Technologies (Tucson, Ariz.), 4th State (Belmont, Calif.), Yield Engineering (San Jose, Calif.), Erie Scientific (Portsmouth, N.H.), and AST Products (advanced surface technologies) (Billerica, Mass.).

Acoustic Biosensors

In another embodiment of the invention, an acoustic biosensor is used. Acoustic biosensors measures the binding of a molecule, such as an analyte, to a chemical or biological molecule that is covalently attached to the surface by detecting a change in the resonant oscillating frequency on the biosensor surface caused by a change in deposited mass as a result of the binding of the molecule and/or analyte. The resonant oscillating frequency can be measured, for example, by using piezoresistive devices, mechanical vibrators, such as micromachined cantilevers, membranes, or tuning forks, or surface acoustic wave oscillators.

Electronic Biosensors

In another embodiment of the invention, an electronic biosensor is used. Electronic biosensors measures the binding of a molecule, such as an analyte, to a chemical of biological molecule that is covalently attached to the surface by detecting a change of resistively, for example DC or AC, low or high frequency, capacitance, or inductance on the biosensor surface caused by a change in deposited mass as a result of the binding of the molecule and/or analyte.

Specific Binding Substances and Binding Partners

One or more specific binding substances are immobilized on the one- or two-dimensional grating or cover layer, if present, by for example, physical adsorption or by chemical binding. A specific binding substance can be, for example, a nucleic acid, peptide, polypeptide, protein, antigen, polyclonal antibody, monoclonal antibody, single chain antibody (scFv), F(ab) fragment, F(ab′)₂ fragment, Fv fragment, small organic molecule, biotin cell, virus, bacteria, polymer, peptide solutions, single- or double-stranded DNA solutions, RNA solutions, solutions containing compounds from a combinatorial chemical library, or biological sample. A biological sample can be for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, or prostatitc fluid.

Preferably, one or more specific binding substances are arranged in a microarray of distinct locations on a biosensor. A microarray of specific binding substances comprises one or more specific binding substances on a surface of a biosensor of the invention such that a surface contains many distinct locations, each with a different specific binding substance or with a different amount of a specific binding substance. For example, an array can comprise 1, 10, 100, 1,000, 10,000, or 100,000 distinct locations. Such a biosensor surface is called a microarray because one or more specific binding substances are typically laid out in a regular grid pattern in x-y coordinates. However, a microarray of the invention can comprise one or more specific binding substance laid out in any type of regular or irregular pattern. For example, distinct locations can define a microarray of spots of one or more specific binding substances. A microarray spot can be about 50 to about 500 microns in diameter. A microarray spot can also be about 150 to about 200 microns in diameter. One or more specific binding substances can be bound to their specific binding partners.

A microarray on a biosensor of the invention can be created by placing microdroplets of one or more specific binding substances onto, for example, an x-y grid of locations on a one- or two-dimensional grating or cover layer surface. When the biosensor is exposed to a test sample comprising one or more binding partners, the binding partners will be preferentially attracted to distinct locations on the microarray that comprise specific binding substances that have high affinity for the binding partners. Some of the distinct locations will gather binding partners onto their surface, while other locations will not.

A specific binding substance specifically binds to a binding partner that is added to the surface of a biosensor of the invention. A specific binding substance specifically binds to its binding partner, but does not substantially bind other binding partners added to the surface of a biosensor. For example, where the specific binding substance is an antibody and its binding partner is a particular antigen, the antibody specifically binds to the particular antigen, but does not substantially bind other antigens. A binding partner can be, for example, a nucleic acid, polypeptide, antigen, polyclonal antibody, monoclonal antibody, single chain antibody (scFv), F(ab) fragment, F(ab′)₂ fragment, Fv fragment, small organic molecule, cell, virus, bacteria, polymer, peptide solutions, single- or double-stranded DNA solutions, RNA solutions, solutions containing compounds from a combinatorial chemical library and biological sample. A biological sample can be, for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, and prostatitc fluid.

One example of a microarray of the invention is a nucleic acid microarray, in which each distinct location within the array contains a different nucleic acid molecule. In this embodiment, the spots within the nucleic acid microarray detect complementary chemical binding with an opposing strand of a nucleic acid in a test sample.

While microtiter plates are the most common format used for biochemical assays, microarrays are increasingly seen as a means for maximizing the number of biochemical interactions that can be measured at one time while minimizing the volume of precious reagents. By application of specific binding substances with a microarray spotter onto a biosensor of the invention, specific binding substance densities of 10,000 specific binding substances/in₂ can be obtained. By focusing an illumination beam to interrogate a single microarray location, a biosensor can be used as a label-free microarray readout system.

Immobilization of One or More Specific Binding Substances

Immobilization of one or more binding substances onto a biosensor is performed so that a specific binding substance will not be washed away by rinsing procedures, and so that its binding to binding partners in a test sample is unimpeded by the biosensor surface. Several different types of surface chemistry strategies have been implemented for covalent attachment of specific binding substances to, for example, glass for use in various types of microarrays and biosensors. These same methods can be readily adapted to a biosensor of the invention. Surface preparation of a biosensor so that it contains the correct functional groups for binding one or more specific binding substances is an integral part of the biosensor manufacturing process.

As used herein, the term “chemical or biological molecules” refers to any chemical or biological molecules that can by attached to the one-or more amine containing polymers. Chemical or biological molecules can be selected from the group consisting of proteins, peptides, polypeptides, nucleotides, polynucleotides, small molecules, biotin, cells, fractionated cells, cells extracts, cell fractions, and parts of cells.

As used herein, the terms protein, peptide and polypeptide refer to a polymer of amino acid residues. The terms also apply to amino acid polymers in which one or more amino acids are chemical analogues of corresponding naturally-occurring amino acids, including amino acids which are modified by post-translational processes (e.g., glycosylation and phosphorylation). The term “protein,” as used herein, means any protein, including, but not limited to peptides, enzymes, glycoproteins, hormones, receptors, antigens, antibodies, growth factors, etc., without limitation.

The term “polypeptide” refers to a polymer of amino acids without regard to the length of the polymer; thus, peptides, oligopeptides, and proteins are included within the definition of polypeptide. This term refers to both naturally occurring polypeptides and synthetic polypeptides. This term can include chemical or post-expression modifications of the polypeptide. Therefore, for example, modifications to polypeptides which include the covalent attachment of glycosyl groups, acetyl groups, phosphate groups, lipid groups and the like are expressly encompassed by the term polypeptide. A chemically modified polypeptides includes polypeptides where an identification or capture tag has been incorporated into the polypeptide. The natural or other chemical modifications, such as those listed in example above, can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, hydrogenation, iodination, methylation, myristoylation, oxidation, pegylation, proteolytic processing, phosphorylation, prenylation, racemization, selenylation, sulfation, transfer-RNA mediated addition of amino acids to proteins such as arginylation, and ubiquitination. (See, for instance, Proteins—Structure and Molecular Properties, 2nd Ed., T. E. Creighton, W. H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)). Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring. The polypeptide may be naturally occurring or synthetic

One or more specific binding substances can be attached to a biosensor surface by physical adsorption (i.e., without the use of chemical linkers) or by chemical binding (i.e., with the use of chemical linkers). Chemical binding can generate stronger attachment of specific binding substances on a biosensor surface and provide defined orientation and conformation of the surface-bound molecules.

For the detection of binding partners at concentrations less than about ˜0.1 ng/ml, it is preferable to amplify and transduce binding partners bound to a biosensor into an additional layer on the biosensor surface. The increased mass deposited on the biosensor can be easily detected as a consequence of increased optical path length. By incorporating greater mass onto a biosensor surface, the optical density of binding partners on the surface is also increased, thus rendering a greater resonant wavelength shift than would occur without the added mass. The addition of mass can be accomplished, for example, enzymatically, through a “sandwich” assay, or by direct application of mass to the biosensor surface in the form of appropriately conjugated beads or polymers of various size and composition. This principle has been exploited for other types of optical biosensors to demonstrate sensitivity increases over 1500× beyond sensitivity limits achieved without mass amplification. See, e.g., Jenison et al., “Interference-based detection of nucleic acid targets on optically coated silicon,” Nature Biotechnology, 19: 62-65, 2001.

As an example, an NH₂-functionalized biosensor surface can have a specific binding substance comprising a single-strand DNA captured probe immobilized on the surface. The capture probe interacts selectively with its complementary target binding partner. The binding partner, in turn, can be designed to include a sequence or tag that will bind a “detector” molecule. A detector molecule can contain, for example, a linker to horseradish peroxidase (HRP) that, when exposed to the correct enzyme, will selectively deposit additional material on the biosensor only where the detector molecule is present. Such a procedure can add, for example, 300 angstroms of detectable biomaterial to the biosensor within a few minutes.

A “sandwich” approach can also be used to enhance detection sensitivity. In this approach, a large molecular weight molecule can be used to amplify the presence of a low molecular weight molecule. For example, a binding partner with a molecular weight of, for example, about 0.1 kDa to about 20 kDa, can be tagged with, for example, succinimidyl-6-[a-methyl-a-(2-pyridyl-dithio)toluamido]hexanoate (SMPT), or dimethylpimelimidate (DMP), histidine, or a biotin molecule. Where the tag is biotin, the biotin molecule will binds strongly with streptavidin, which has a molecular weight of 60 kDa. Because the biotin/streptavidin interaction is highly specific, the streptavidin amplifies the signal that would be produced only by the small binding partner by a factor of 60.

Detection sensitivity can be further enhanced through the use of chemically derivatized small particles. “Nanoparticles” made of colloidal gold, various plastics, or glass with diameters of about 3-300 nm can be coated with molecular species that will enable them to covalently bind selectively to a binding partner. For example, nanoparticles that are covalently coated with streptavidin can be used to enhance the visibility of biotin-tagged binding partners on the biosensor surface. While a streptavidin molecule itself has a molecular weight of 60 kDa, the derivatized bead can have a molecular weight of any size, including, for example, 60 KDa. Binding of a large bead will result in a large change in the optical density upon the biosensor surface, and an easily measurable signal. This method can result in an approximately 1000× enhancement in sensitivity resolution.

Methods of Using Biosensors

Biosensors of the invention can be used to study one or a number of specific binding substance/binding partner interactions in parallel. Binding of one or more specific binding substances to their respective binding partners can be detected, without the use of labels, by applying one or more binding partners to the biosensor that have one or more specific binding substances immobilized on their surfaces. For example, an SWS biosensor is illuminated With light and a maxima in reflected wavelength, or a minima in transmitted wavelength of light is detected from the biosensor. If one or more specific binding substances have bound to their respective binding partners, then the reflected wavelength of light is shifted as compared to a situation where one or more specific binding substances have not bound to their respective binding partners. Where a SWS biosensor is coated with an array of distinct locations containing the one or more specific binding substances, then a maxima in reflected wavelength or minima in transmitted wavelength of light is detected from each distinct location of the biosensor.

In one embodiment of the invention, a variety of specific binding substances, for example, antibodies, can be immobilized in an array format onto a biosensor of the invention. The biosensor is then contacted with a test sample of interest comprising binding partners, such as proteins. Only the proteins that specifically bind to the antibodies immobilized on the biosensor remain bound to the biosensor. Such an approach is essentially a large-scale version of an enzyme-linked immunosorbent assay; however, the use of an enzyme or fluorescent label is not required.

The activity of an enzyme can be detected by applying one or more enzymes to a biosensor to which one or more specific binding substances have been immobilized. For example, the biosensor is washed and illuminated with light. The reflected wavelength of light is detected from the biosensor. Where the one or more enzymes have altered the one or more specific binding substances of the biosensor by enzymatic activity, the reflected wavelength of light is shifted.

Additionally, a test sample, for example, cell lysates containing binding partners can be applied to a biosensor of the invention, followed by washing to remove unbound material. The binding partners that bind to a biosensor can be eluted from the biosensor and identified by, for example, mass spectrometry. Optionally, a phage DNA display library can be applied to a biosensor of the invention followed by washing to remove unbound material. Individual phage particles bound to the biosensor can be isolated and the inserts in these phage particles can then be sequenced to determine the identity of the binding partner.

For the above applications, and in particular proteomics applications, the ability to selectively bind material, such as binding partners from a test sample onto a biosensor of the invention, followed by the ability to selectively remove bound material from a distinct location of the biosensor for further analysis is advantageous. Biosensors of the invention are also capable of detecting and quantifying the amount of a binding partner from a sample that is bound to a biosensor array distinct location by measuring the shift in reflected wavelength of light. For example, the wavelength shift at one distinct biosensor location can be compared to positive and negative controls at other distinct biosensor locations to determine the amount of a binding partner that is bound to a biosensor array distinct location.

### EXAMPLE 1

### Fabrication of a SWS Biosensor

The detailed manufacture process of the SWS biosensor has been described previously. See, e.g., Cunningham B. et al., Sensor and Actuators B 6779, 1-6 (2002), incorporated herein by reference. Specifically, an optical-grade polymer film was used as a support of SWS sensor. A UV-curable acrylic-based polymer coating was coated onto the film and replicated using a silicon mask that has 96 circles corresponding to the standard format of a 96-well micro-titer plate, which circles form an SWS structure. A UV lamp RC600, provided by Xenon Corporation (Woburn, Mass.), was used to cure the coating after the replication. Subsequently, a titanium dioxide layer and a silicone dioxide layer were deposited onto the top of the surface.

### EXAMPLE 2

### Silanization Using an Epoxy Silane

The fabricated SWS biosensor sheets were immersed in 50 mLs of 50 parts per million NaOH in deionized water for 20 minutes, and then rinsed with a large amount of deionized water. A silane solution was prepared using 4 mL 3-glycidoxypropyltrimethoxysilane (Z-6040), provided by Dow Corning (Midland, Mich.), and 196 mL of a solvent mixture containing 95% ethanol, 5% deionized water and 0.1 mL acetic acid. The silane solution was aged for 15 minutes prior to silanization. The cleaned SWS biosensor sheets were immersed in the silane solution for 1 minute. They were then rinsed three times with 200 mL isopropanol. The SWS biosensors were dried using a centrifuge and cured in a 65% relative humidity chamber for 18 hour.

### EXAMPLE 3

### Surface Grafting with Polyethylenimine

Polyethylenimine (PEI), provided in a solution of 50% in water by Aldrich Chemical (Milwaukee, Wis.), was diluted to 20%, 15%, 10%, 10%, 5% and 1.5% and adjusted to pH 8.0 with concentrated hydrogen chloride. The silanized SWS biosensor sheets described in Example 2 were immersed in the prepared PEI solutions for 18 hours, and were rinsed first using deionized water, then rinsed using 3×PBS plus 0.5% Tween 20, and were finally rinsed using deionized water.

### EXAMPLE 4

### Measurement of Amine Group Density on Surface

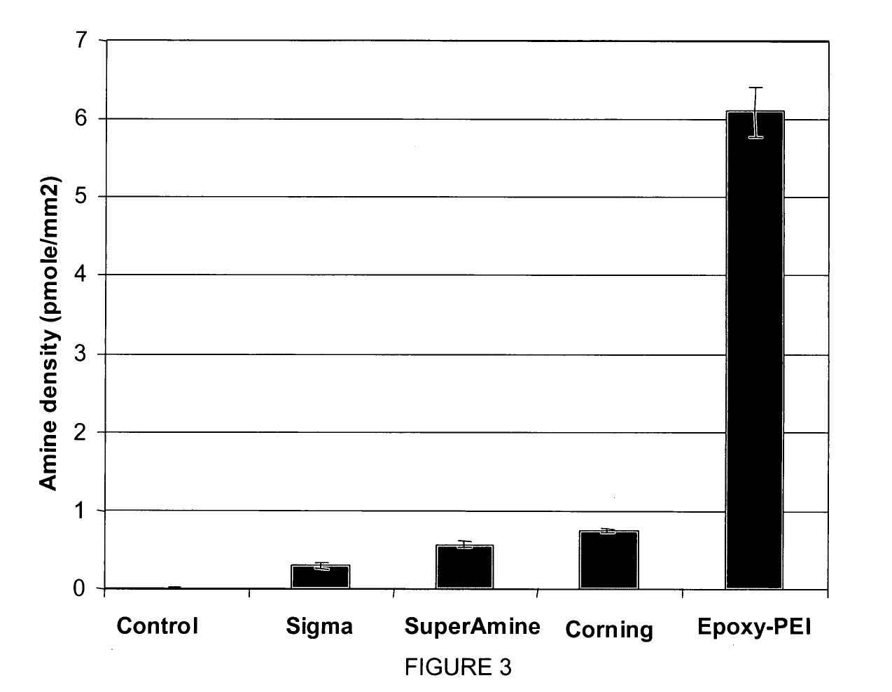

The SWS biosensor sheets described in Example 3 were cut into 25×75 mm size. In order to measure the amine group density on various surfaces, five groups of slides, consisting of the cut SWS biosensor sheets, Corning GAPS II amino-silane coated slides from Corning (Corning, N.Y.), Arryit SuperAmine slides from TeleChem International (Sunnyvale, Calif.), Sigma Silane-Prep amine slides from Sigma (St Louis, Mo.), and cleaned glass slides as a control, were placed in five square dishes respectively. The measurements were carried out in triplet. 20 mL of 0.1 mM Sulfosuccinidyl-4-O-(4,4′-dimethoxytrityl)-butyrate (Sulfo-SDTB) in 50 mM sodium bicarbonate buffer, pH 8.5 was prepared and poured immediately into each of the dishes. The dishes were shaken for 30 minutes and the samples were subsequently dried. 19×60 mm opening gasket chambers from MJ Research (Watertown, Mass.) were placed onto each of the samples. 400 μL of 30% perchloric acid was added to each of the chambers. The samples were then shaken on a shaker for 10 minutes. 200 μL of the resulting solution was measured at an absorbance of 495 nm using a plate reader SpectraMax Plus 384 from Molecular Devices (Sunnyvale, Calif.). The concentration of the product was calculated based on the measured absorbance using an extinction coefficient of 70,000 M⁻¹ cm⁻¹. Gaur, R. K. and Gupt, K. C., Anal. Biochem. 180, 253-258 (1989). The density of the amine group on the samples is depicted in FIG. 3. This experimental result reflects the density of amine groups on the surfaces that was accessible and able to react with the relatively large sized Sulfo-SDTB (MW 605.59).

### EXAMPLE 5

### Measurement of PEI Layer's Thickness Using Ellipsometer

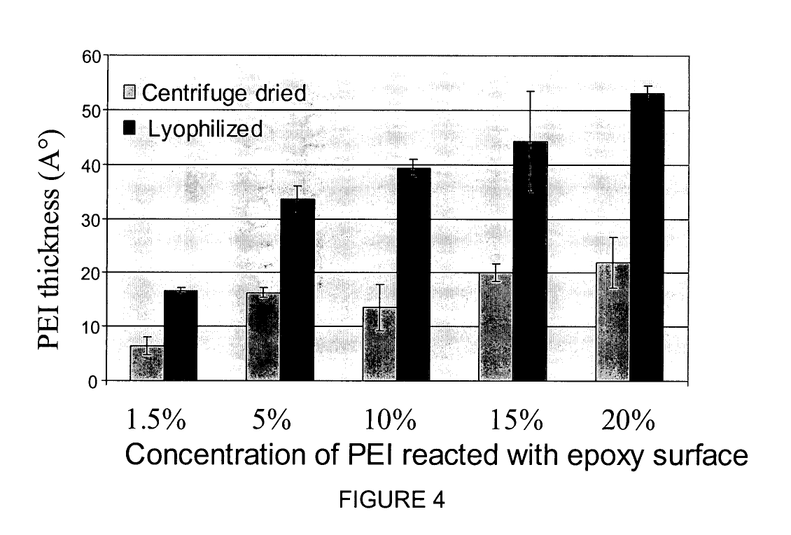

Silicon wafers GH503-3 provided by SI-TECH (Geneva, Ill.) were cut into 2×3 cm pieces. The 2×3 cm pieces were cleaned by dipping 10% NaOH in deionized water for 20 minutes then rinsing with a large amount of deionized water. After drying, the silicon pieces were silanized using the epoxy silane Z-6040 following the protocol described in Example 2. The five group of silanized silicon pieces were immersed in 50 mL of 20%, 15%, 10%, 5% and 1.5% PEI in deionized water, pH 8.0, for 18 hours in triplet, then rinsed with large amount of water. The five pieces of the samples from each group were dried using a centrifuge. Another five pieces of the samples from each of the groups were frozen in liquid nitrogen, and then dried in a lyophilizer. The PEI thickness of the two sets of the samples was measured using an ellipsometer Gaertner L116A manufactured by Gaertner Scientific Corp. (Skokie, Ill.). The thickness indicates that the thicker PEI layer was grafted onto the epoxy surface when the higher concentration of PEI was employed (see FIG. 4). Compared to the centrifuge dried samples, the lyophilized samples in the same group showed greater thickness. Although the same amounts of PEI Were grafted onto the surfaces of both lyophilized and centrifuged samples, the thicker PEI layer of the lyophilized samples was observed. That indicated that PEI layer on lyophilized samples was porous and lyophilizing froze some polymeric structure of grafted PEI in the aqueous medium. This showed that the PEI polymer chains on the surface were extended more in the aqueous medium compared to the PEI layer in dried form. The extended PEI polymer chain formed an accessible layer on the surface that has thickness of 50 Å at least. The thickness of the PEI layer on the surface in aqueous medium established that the structure was a three-dimensional polymer substrate, expected to reduce steric hindrance as compared to a two-dimensional surface.

### EXAMPLE 6

### Surface Characterization Using X-ray Photoelectron Spectroscopy (XPS)

The surface elements of the samples prepared in Example 3 were analyzed using XPS. 55° of takeoff angle was selected and approximately 5 nm top surface layer was analyzed. The nitrogen was only provided by PEI and was used to estimate PEI amount on the surfaces. Table 1 shows that the nitrogen content increased as the higher concentration of PEI was used in the grafting reaction.

### EXAMPLE 7

### Immobilization of Biotin Onto PEI Sensor Surface

The silanized-sensor sheet in Example 2 was attached to the bottom of a bottomless 96-well plate. 200 uL of 15% PEI in deionized water, pH 8.0, was placed in 3×6 wells and removed after 18 hours. The wells were rinsed according to the protocol described in Example 3. The rest of epoxy surface wells were used as control in later experiments.

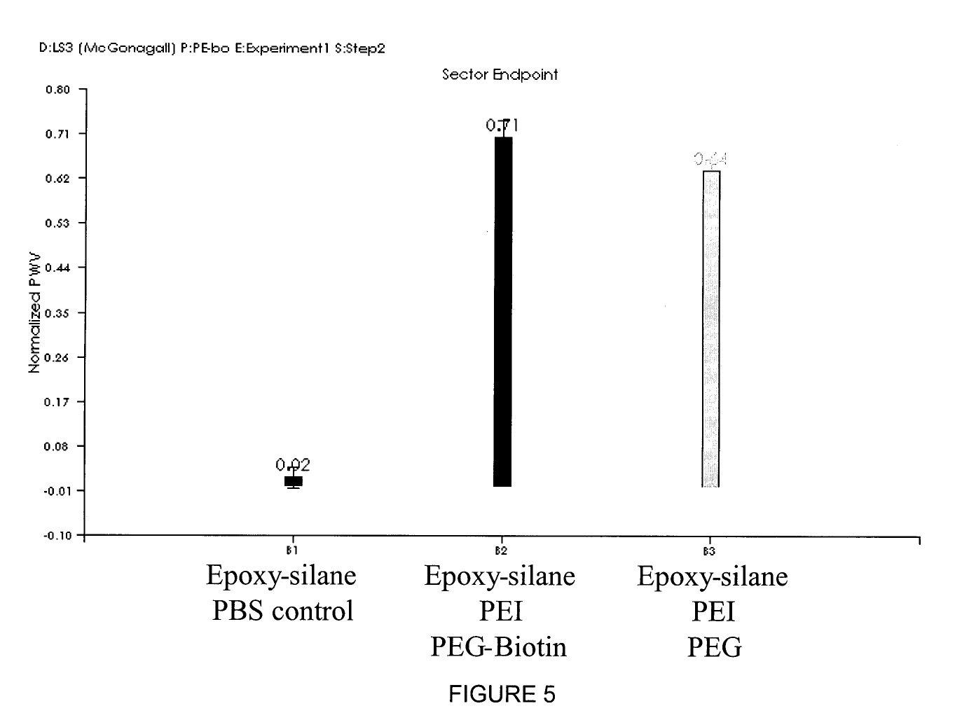

1 mg/mL Biotin-PEG-CO2-NHS (FW 3400) and 1 mL/mL of nPEG-CM-HBA-NHS (FW 3400) were prepared in 1×PBS buffer pH 7.4 (“PBS”). Both of the reagents were provided by Nektar Therapeutics, San Carlos, Calif. 200 mL of the two prepared reagent solutions and PBS as control were placed in the 6 wells for each of the three groups and rinsed with PBS three times, then measured using a 96-well plate reader (SRU Biosystems, Woburn, Mass.). The detection response of the amount of molecule attached to the surface, PWV shift, was described previously. Cuningham B., et al., Sensor and Actuators B 6779:1-6 (2002). The PWV for the three groups are showed in FIG. 5. Both Biotin-PEG-CO2-NHS and mPEG-CM-HBA-NHS immobilized to the sensor surface using the reaction of the amine on the PEI and NHS ester from the two reagents compared to the control.

### EXAMPLE 8

### Binding Streptavidin Onto Biotinylated PEI Surface

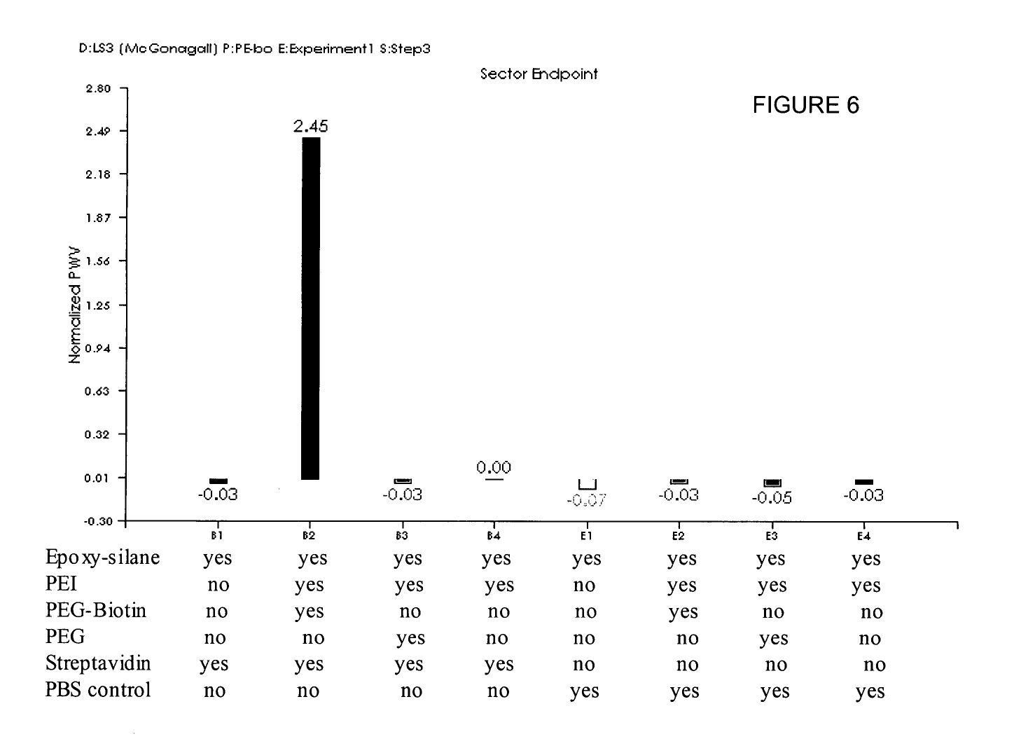

100 ug/mL of Streptavidin (SA) was prepared in PBS using SA10, provided by Prozyme Inc. (San Leandro, Calif.). 200 mL of SA solution and PBS control was added respectively in three wells in each of the groups in Example 6 and epoxy surface wells. After 60 minutes of incubation at room temperature, the experimental wells were rinsed using PBS three times. The response of SA binding was measured using the 96-well plate reader and is shown in FIG. 6. SA was bound to the biotin immobilized on the surface suing PEI significantly compared to the other seven control groups.

### EXAMPLE 9

### Surface Grafting With Polyvinylamine

Polyvinylamine (PVA), provided in a solution of 20% in water by BASF Corporation (Mount Olive, N.J.) under the trade name LUPAMINO 9095, was diluted to 15% using water, adjusted pH to 7.4 using concentrated hydrogen chloride. 100 μL of the PVA solution was added to each well of a 96-well plate. The 96-well plate was fabricated by attaching the silanized SWS biosensor sheet as described in Example 2 to a bottomless 96-well plate. The PVA solution was allowed to react to the epoxy-silane-treated surface overnight at room temperature in the wells, and was removed the second day. The wells were washed with water three times and emptied. 100 μL of 25% glutaraldehyde solution in water was added to each well and removed after a 2 hr incubation. The wells were rinsed with water three times and PBS buffer pH 7.4 three times and stored in PBS.

### EXAMPLE 10

### Immobilization of Streptavidin on the Polyvinylamine-functionalized SWS Biosensor Surface

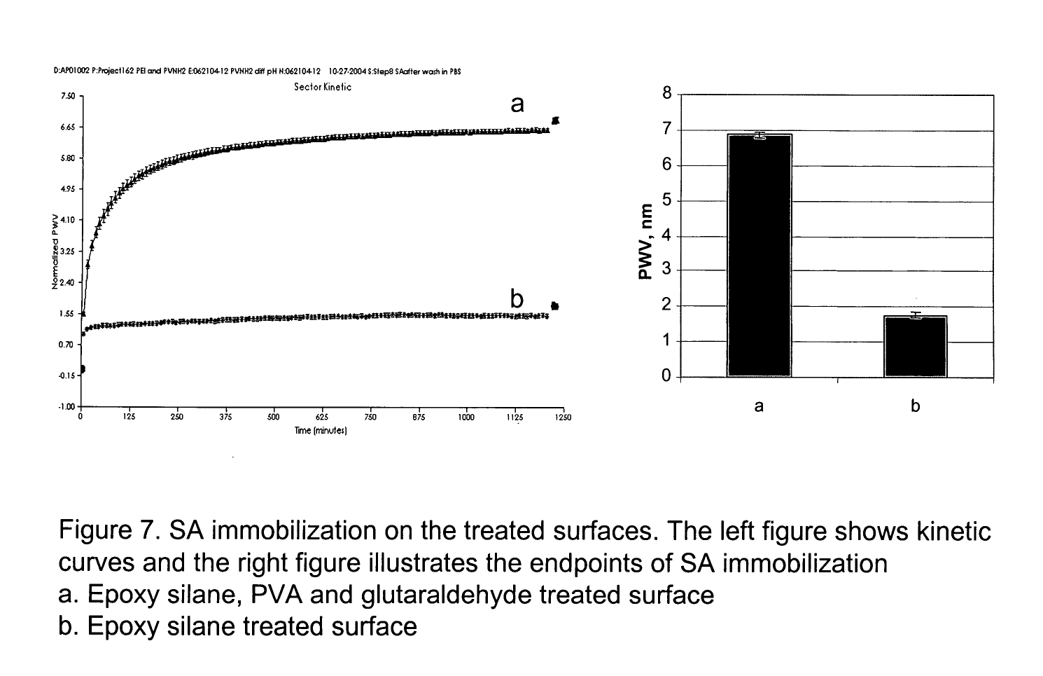

50 μL of a 0.5 mg/mL SA solution prepared in 5 mM sodium phosphate buffer pH 7.4 using SA10, provided by Prozyme Inc. (San Leandro, Calif.) was added into each of the empty wells of the plate as described in Example 9. The plate was placed on a 96-well plate reader (SRU Biosystems, Woburn, Mass.) to observe the response of streptavidin immobilization, which is shown in FIG. 7 left. After 20 hours of the immobilization, the SA solution was removed and the wells were washed using PBS buffer pH 7.4. The endpoints of SA immobilization on the functionalized wells through PVA and control are shown in FIG. 7 right.

### EXAMPLE 11

### Biotin Response to Immobilized SA on the Treated Surface

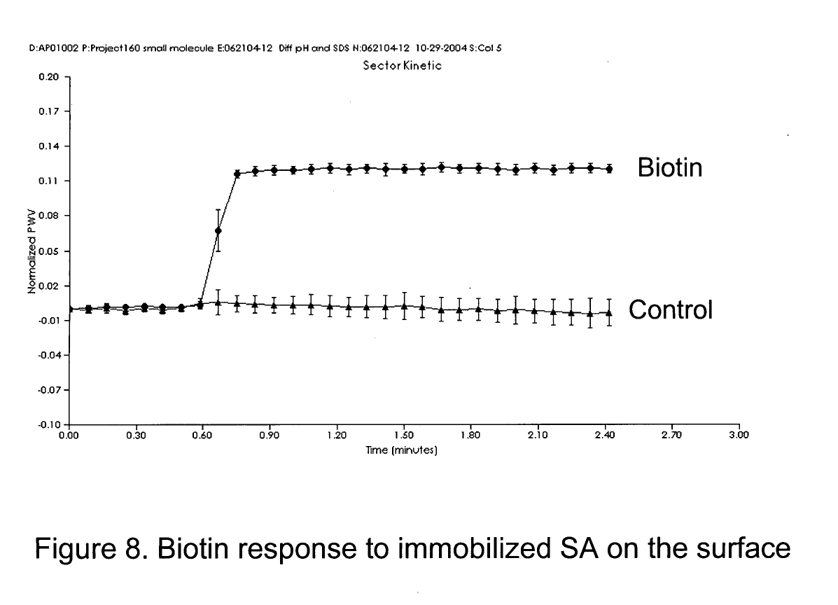

A solution of 0.05 mg/mL biotin (Cat # B0301), provided by Sigma (Milwaukee, Wis.) in PBS buffer pH 7.4, was added into four wells of the SA-attached surface, prepared as described in Example 10, incubated for 0.5 hr to saturate the interaction of biotin and SA. The wells were rinsed three times with PBS Buffer, then emptied. The four biotin-treated wells were used as control. 90 μL of PBS buffer was placed in four different SA-attached empty wells and the four control wells. After 0.6 min baseline reading, 10 μL of 0.5 μg/mL in PBS buffer was added into the four biotin-treated wells and the four control wells. The biotin response, shown in FIG. 8, indicates that immobilized SA remained active.

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims.

1. A method for preparing a high-density amine-functionalized surface, comprising:
(a) treating a surface with epoxy silane to form an epoxy-functional surface; and
(b) attaching one or more amine-containing polymers to the epoxy-functional surface by adding a solution comprising one or more amine-containing polymers to the epoxy-functional surface under conditions where one or more amine-containing polymers react with the epoxy-functional surface; whereby a high-density amine-functionalized surface is formed.

2. The method of claim 1, further comprising the step of covalently attaching one or more chemical or biological molecules to the one or more amine-containing polymers attached to the surface.

3. A biosensor comprising the high-density amine-functionalized surface.

4. The biosensor of claim 3, wherein the biosensor is an optical sensor.

5. The biosensor of claim 4, wherein the biosensor is a calorimetric resonant biosensor.

6. The biosensor of claim 3, wherein the biosensor is an acoustic biosensor.

7. The biosensor of claim 3, wherein the biosensor is an electric biosensor.

8. The method of claim 1, wherein the one or more amine-containing polymers are the same or different.

9. The method of claim 1, wherein the one or more amine-containing polymers are the same.

10. The method of claim 1, wherein the one or more amine-containing polymers comprise primary amines.

11. The method of claim 1, wherein the one or more amine-containing polymers comprise secondary amines.

12. The method of claim 1, wherein the one or more amine-containing polymers comprise primary and secondary amines.

13. The method of claim 1, wherein the one or more amine-containing polymers comprise polyethylenimine.

14. The method of claim 1, wherein the one or more amine-containing polymers consist of polyethylenimine.

15. The method of claim 1, wherein the one or more amine-containing polymers comprise polyvinylamine.

16. The method of claim 1, wherein the one or more amine-containing polymers consist of polyvinylamine.

17. The method of claim 2, wherein the chemical or biological molecules are selected from the group consisting of proteins, peptides, polypeptides, nucleotides, polynucleotides, small molecules, biotin, cells, fractionated cells, cells extracts, cell fractions, and parts of cells.

18. The method of claim 17, wherein the chemical or biological molecules comprise a protein.

19. The method of claim 18, wherein the protein is an enzyme.

20. The method of claim 18, wherein the protein is an antibody.

21. The method of claim 18, wherein the protein is selected from the group consisting of avidin or streptavidin.

22. The method of claim 2, wherein the chemical or biological molecules comprise a peptide.

23. The method of claim 2, wherein the chemical or biological molecules comprise a polynucleotide.

24. The method of claim 2, wherein the chemical or biological molecules comprise a cell, fractionated cell, cells extract, cell fraction, or part of a cell.

25. The method of claim 2, wherein the chemical or biological molecules comprise a small molecule.

26. The method of claim 2, wherein the chemical or biological molecules comprise biotin.

27. A high-density amine-functionalized polymeric matrix, comprising one or more amine-containing polymers covalently attached to a surface through a functional epoxy, wherein the amine-containing polymers are the same or different, and wherein the amine-containing polymers comprise two or more amine groups.

28. The high-density amine-functionalized polymeric matrix of claim 27, wherein the amine-containing polymers comprise three or more amine groups.

29. The high-density amine-functionalized polymeric matrix of claim 27, wherein the amine-containing polymers comprise polyethylenimine.

30. The high-density amine-functionalized polymeric matrix of claim 27, wherein the amine-containing polymers comprise polyvinylamine.

31. A method of immobilizing biomolecules on a surface, comprising contacting biomolecules with the high-density amine-functionalized surface of claim 1, whereby the biomolecules are immobilized.

32. A method of immobilizing biomolecules on a surface, comprising contacting biomolecules with the high-density amine-functionalized surface of claim 5, whereby the biomolecules are immobilized.

33. A method of immobilizing biomolecules on a surface, comprising contacting biomolecules with the high-density amine-functionalized surface of claim 13, whereby the biomolecules are immobilized.

34. A method of immobilizing biomolecules on a surface, comprising contacting biomolecules with the high-density amine-functionalized surface of claim 14, whereby the biomolecules are immobilized.

35. A biosensor comprising a high-density amine-functionalized surface, wherein the high-density amine-functionalized surface is prepared by the method comprising:
(a) treating a surface with epoxy silane to form an epoxy-functional surface; and
(b) attaching one or more amine-containing polymers to the epoxy-functional surface by adding a solution comprising one or more amine-containing polymers to the epoxy-functional surface under conditions where one or more amine-containing polymers react with the epoxy-functional surface; whereby a high-density amine-functionalized surface is formed.

36. The biosensor of claim 35, wherein the biosensor is an optical sensor.

37. The biosensor of claim 36, wherein the biosensor is a calorimetric resonant biosensor.

38. The biosensor of claim 35, wherein the biosensor is an acoustic biosensor.

39. The biosensor of claim 35, wherein the biosensor is an electric biosensor.

40. The method of claim 1, wherein the surface is a plastic surface.

41. The method of claim 40, further comprising the step of covalently attaching one or more chemical or biological molecules to the one or more amine-containing polymers attached to the surface.

42. The biosensor of claim 3, wherein the surface is a plastic surface.

43. The biosensor of claim 35, wherein the surface is a plastic surface.
